(19) United States
(12) Patent Application Publication
(10) Pub. No.: US 2017/0014828 A1
(43) Pub. Date: Jan. 19, 2017

(54) ORGANIC GARBAGE AND KITCHEN WASTE PROCESSING ASSEMBLY

(71) Applicant: YA JYUE CHEN, TAICHUNG CITY (TW)

(72) Inventor: YA JYUE CHEN, TAICHUNG CITY (TW)

(21) Appl. No.: 15/009,827

(22) Filed: Jan. 29, 2016

(30) Foreign Application Priority Data
Jul. 13, 2015 (CN) 201510407966.1

(51) Int. Cl. B02C 13/288; B02C 13/06; C05F 9/02; A61L 11/00

(52) U.S. Cl. CPC B02C 13/288; A61L 11/00; B02C 13/06; C05F 9/02; A23K 10/37

(57) ABSTRACT

A processing assembly for processing kitchen organic garbage and waste of farms includes a tank with a grinder located therein. The grinder grinds the garbage and waste by blades and rotatable blades. A transition can is connected between the tank and a deodorant pool which has deodorant bacteria received therein to remove odors. A one-way filter is connected between the transition can and the deodorant pool. A vacuum pump is connected between the tank and the transition can to suck air in the tank to the deodorant pool and to separate water from the vapor air. The water is stored in the transition can. The volume and weight of the garbage and waste are reduced and no pollution is created to the air. Hard stuff from the organic waste is ground into small pieces without damage to the blades.

### BACKGROUND OF THE INVENTION

1. Fields of the Invention

The present invention relates to a processing assembly for processing kitchen organic garbage and waste of farms.

2. Descriptions of Related Art

The organic garbage and kitchen waste generated from farms mostly are vegetables, fruits, plants, bones, sea shell etc., which contains a certain amount of liquid and can easily generate odors and attract flies.

These kitchen organic garbage and waste of farms that are collected independently can be re-used, such as in the form of organic soil fertilizer, animal feed to create alternative value. By this way, the conventional ways to deal with the garbage such as landfill sites and garbage incineration plants can be reduced.

TW 1366557 discloses a machine for processing organic garbage and kitchen waste generated from farms does not have the function of removing odors from it so that the problems of pollution to air are obvious. Besides, the hard stuff such as coconut husk, bones and sea shells cannot be properly grounded so that the organic fertilizer made from the processed organic garbage and kitchen waste cannot have high quality.

The present invention intends to provide a processing assembly for processing kitchen organic garbage and waste of farms, and the processing assembly eliminates the shortcomings mentioned above.

### SUMMARY OF THE INVENTION

The present invention relates to a processing assembly for processing kitchen organic garbage and waste of farms, and the processing assembly comprises a tank having a cover unit and a door respectively connected to outside thereof. A grinder is located in the tank and driven by a driving device. The grinder has fixed blades and rotatable blades, wherein the fixed blades extend from the inside of the tank and are arranged in a spiral form. The rotatable blades are connected to a shaft driven by the driving device. A deodorant pool has deodorant bacteria received therein. A transition can is connected between the tank and the deodorant pool. A one-way filter is connected between the transition can and the deodorant pool. A vacuum pump is connected between the tank and the transition can so as to suck vapor air in the tank to the deodorant pool and to separate water from the vapor. The water is stored in the transition can.

Preferably, the cover unit includes a bottom part and a top part, wherein the bottom part is connected to the tank. The top part has a cover, an arm and a pivotal seat, wherein the pivotal seat is connected to one side of the bottom part. The arm is pivotably connected to the pivotal seat and has a resilient member connected thereto. The lower end of the resilient member is connected to the cover.

Preferably, the bottom part has multiple locking units connected thereto.

Preferably, each of the locking units has a loop which is connected to a bolt. The lower end of the loop is connected to a base, and the lower end of the bolt is connected to a locking member. A spring is connected between the base and the locking member. The locking member is connected to the bottom part. The base is engaged with the periphery of the top part. The loop is rotated to secure the cover of the top part.

Preferably, the rotatable blades are connected with a crescent blade and a hammer.

Preferably, gaps are formed between the fixed blades, the hammers and the crescent blade so as to grind garbage and waste.

Preferably, a scraper is connected to each of two inner end faces of the tank.

Preferably, the deodorant pool has a cool water path, an inlet for introducing cool water, and an electric valve for releasing water in the deodorant pool. The inlet is connected to the vacuum pump to cool the vacuum pump.

Preferably, the electric valve opens when the temperature of the water in the deodorant pool is higher than 45 degrees Celsius; the water in the deodorant pool drains out from the electric valve. The inlet introduces cool water into the deodorant pool and cools down the deodorant pool.

The primary object of the present invention is to provide a processing assembly for processing kitchen organic garbage and waste of farms, wherein more than 90% of the volume and weight of the garbage and waste are reduced. The odors can be removed by using the transition can and the deodorant pool. Coconut husk, bones and sea shells in the garbage and waste are ground into small pieces without damage to the blades.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the processing assembly of the present invention;

FIG. 2 shows the processing assembly of the present invention wherein the casing and cover unit are removed;

FIG. 3 shows the grinder of the processing assembly of the present invention;

FIG. 4 shows the locking unit of the processing assembly of the present invention, and

FIG. 5 shows slow chart of the processing assembly of the present invention.

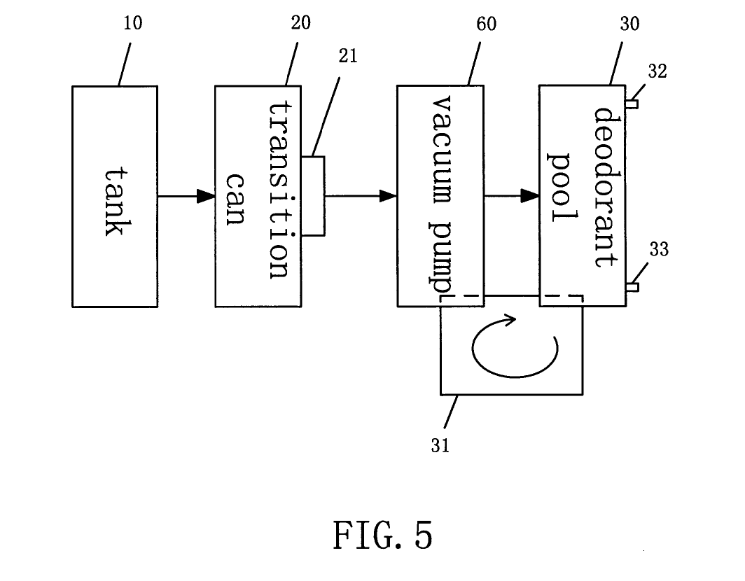

### DETAILED DESCRIPTION OF THE PREFERRED EMBODIMENT

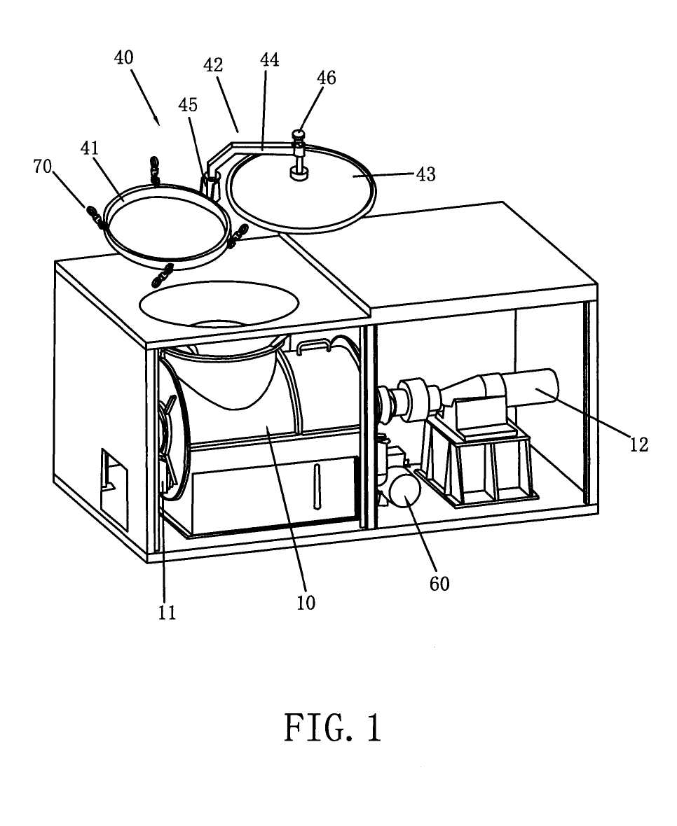

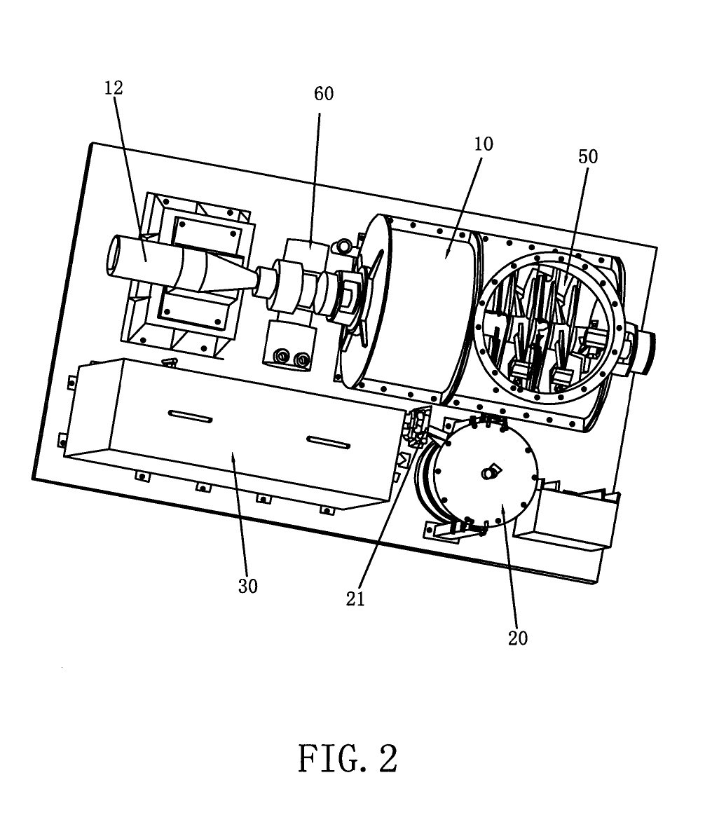

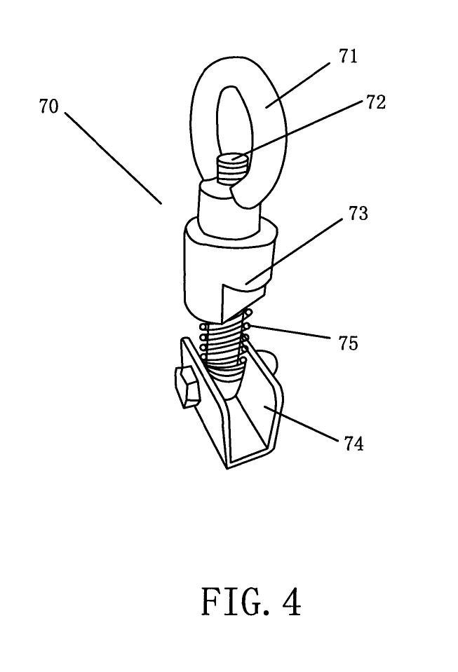

Referring to FIGS. 1 to 4, the processing assembly of the present invention comprises a tank 10, a transition can 20 and a deodorant pool 30. The tank 10 is a sealed tank and a cover unit 40 is connected to the top thereof, and a door 11 is connected to one end thereof. The door 11 is used to removal of the processed garbage and waste. A grinder 50 is located in the tank 10.

The transition can 20 is connected between the tank 10 and the deodorant pool 30, and a vacuum pump 60 is connected between the tank 10 and the transition can 20. A one-way filter 21 is connected between the transition can 20 and the deodorant pool 30. The one-way filter 21 filters the odors and water from the vapor air, dry air is separated from the vapor air, and enters into the vacuum pump 60. By the vacuum pump 60, the air in the tank 10 and the transition can 20 is sucked into the deodorant pool 30. The tank 10 and the transition can 20 are kept as vacuum status.

The deodorant pool 30 has deodorant bacteria received therein so as to remove the odors and sterilize the air and liquid from the tank 10. The deodorant pool 30 has a cool water path 31, an inlet 32 for introducing cool water, and an electric valve 33 for releasing water in the deodorant pool 30. Preferably, there is a drain pipe is connected to about ⅘ height from the bottom of the deodorant pool 30. The inlet 32 is connected to the vacuum pump 60 so as to cool the vacuum pump 60 and increase the operation efficiency of the vacuum pump 60. The electric valve 33 opens when a temperature of the water in the deodorant pool 30 is higher than 45 degrees Celsius, the water in the deodorant pool 30 drains out from the electric valve 33. The inlet 32 introduces cool water into the deodorant pool 30 and cools down the deodorant pool 30.

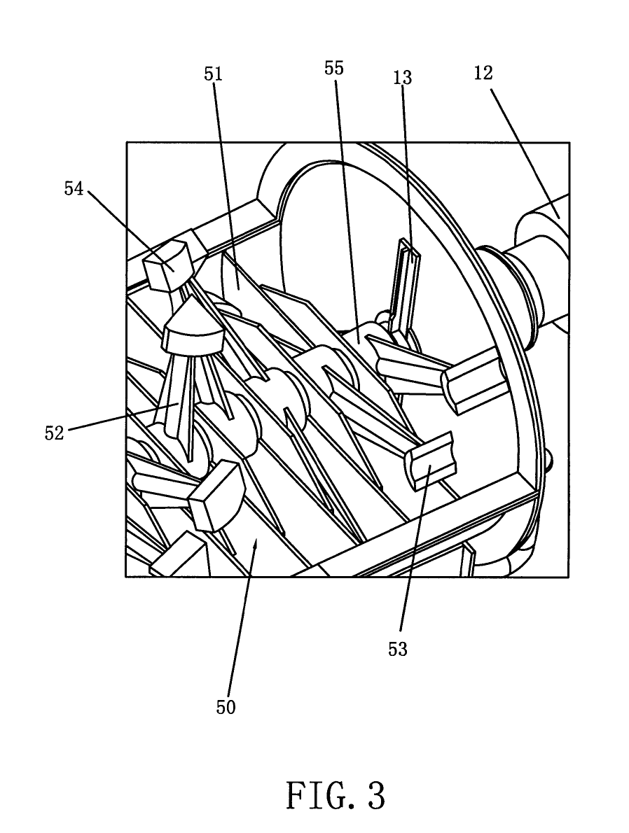

As shown in FIG. 3, the grinder 50 is driven by a driving device 12 and has fixed blades 51 and rotatable blades 52. The fixed blades 51 extend from the inside of the tank 10 and are arranged in a spiral form. The garbage and the waste is pushed outward by the spirally arranged fixed blades 51. The rotatable blades 52 are connected to a shaft 55 driven by the driving device 12. The rotatable blades 52 are connected with a crescent blade 53 and a hammer 54. Gaps are formed between the fixed blades 51, the hammers 54 and the crescent blade 53 so as to grind the bones, shells and wood chips in the garbage and waste. A scraper 13 is connected to each of two inner end faces of the tank 10 so as to scrap remains attached on the inner end faces of the tank 10.

The cover unit 40 has a bottom part 41 and a top part 42, wherein the bottom part 41 is connected to the tank 10. The top part 42 has a cover 43, an arm 44 and a pivotal seat 45. The pivotal seat 45 is connected to one side of the bottom part 41, and the arm 44 is pivotably connected to the pivotal seat 45 so that the arm 44 is pivoted 360 degrees about the pivotal seat 45. A resilient member 46 is connected to the arm 44 and is able to move up and down. The lower end of the resilient member 46 is connected to the cover 43. When the vacuum pump 60 is activated, the cover 43 is sucked downward, the resilient member 46 allows the movement of the cover 43 to move. The cover 43 is connected to the arm 44 so that the cover 43 is shifted when the arm 44 is pivoted.

The bottom part 41 has multiple locking units 70 connected thereto so as to lock the bottom part 41 and the top part 42. Each of the locking units 70 has a loop 71 which is connected to a bolt 72. The lower end of the loop 71 is connected to a base 73. The lower end of the bolt 72 is connected to a locking member 74. A spring 75 is connected between the base 73 and the locking member 74, and the locking member 74 is connected to the bottom part 41. The base 73 is engaged with the periphery of the top part 42, the loop 71 is rotated to secure the cover 43 of the top part 42. When operation the top part 42 and the bottom part 41, the locking member 74 is fixed to the bottom part 41 and the loop 71 is rotated to move the base 73 upward. After the base 73 is moved a distance, the distance between the base 73 and the locking member 74 is larger than the combined thickness of the bottom part 41 and the top part 42, the base 73 is engaged with the periphery of the top part 42 and the locking member 74 has its lower end locks the bottom part 41. The top end of the base 73 is engaged with the top part 42. The spring 75 maintains the engagement, the loop 71 is rotated downward to reduce the distance between the base 73 and the locking member 74, so that the bottom part 41 is tightly connected to the top part 42.

When in use, by loosening the loop 71, the base 73 is pulled upward from the top part 42 to release the connection between the bottom part 41 and the top part 42. The resilient member 46 lifts the cover 43 upward and separates the cover 43 from the bottom part 41. The arm 44 is then rotated an angle to move the top part 42 away from the bottom part 41 so as to open the entrance of the tank 10. The garbage and waste are put into the tank 10 from the entrance. The top part 42 is then moved to the bottom part 41 by swinging the arm 44, the base 73 of each of the locking units 70 is then engaged with the top part 42 and the loop 71 is rotated to tightly connect the bottom part 41 to the top part 42. When the tank 10 is under vacuum status, the top part 42 is sucked downward, and the spring of the resilient member 46 is activated so that the top part 42, the cover 43, the arm 44 and the pivotal seat 45 are not deformed. Therefore, the bottom part 41 is tightly connected to the top part 42 to achieve the vacuum status.

The driving device 12 is then activated, the shaft 55 is rotated by the driving device 12, and the crescent blades 53 and the hammers 54 on the rotatable blades 52 are rotated and cooperate with the fixed blades 53 to grind the garbage and the waste. The vacuum pump 60 sucks the air in the tank 10 and the transition can 20 into the deodorant pool 30 to make the tank 10 and the transition can 20 be vacuum status. During the operation of the grinder 50, the spirally arranged fixed blades 51 push the garbage and the waste toward the door 11. When the door 11 is opened, the ground garbage and the waste are removed from the door 11 and collected.

While we have shown and described the embodiment in accordance with the present invention, it should be clear to those skilled in the art that further embodiments may be made without departing from the scope of the present invention.

What is claimed is:

1. A processing assembly for processing kitchen organic garbage and waste of farms, comprising:
a tank having a cover unit and a door respectively connected to outside thereof;
a grinder located in the tank and driven by a driving device, the grinder having fixed blades and rotatable blades, the fixed blades extending from an inside of the tank and being arranged in a spiral form, the rotatable blades connected to a shaft driven by the driving device;
a deodorant pool having deodorant bacteria received therein;
a transition can connected between the tank and the deodorant pool, a one-way filter connected between the transition can and the deodorant pool, and
a vacuum pump connected between the tank and the transition can so as to suck air in the tank to the deodorant pool and to separate water from the vapor air, the water being stored in the transition can.

2. The processing assembly as claimed in claim 1, wherein the cover unit has a bottom part and a top part, the bottom part is connected to the tank, the top part has a cover, an arm and a pivotal seat, the pivotal seat is connected to one side of the bottom part, the arm is pivotably connected to the pivotal seat and has a resilient member connected thereto, a lower end of the resilient member is connected to the cover.

3. The processing assembly as claimed in claim 1, wherein the bottom part has multiple locking units connected thereto.

4. The processing assembly as claimed in claim 3, wherein each of the locking units has a loop which is connected to a bolt, a lower end of the loop is connected to a base, a lower end of the bolt is connected to a locking member, a spring is connected between the base and the locking member, the locking member is connected to the bottom part, the base is engaged with a periphery of the top part, the loop is rotated to secure the cover of the top part.

5. The processing assembly as claimed in claim 1, wherein the rotatable blades are connected with a crescent blade and a hammer.

6. The processing assembly as claimed in claim 5, wherein gaps are formed between the fixed blades, the hammers and the crescent blade so as to grind garbage and waste.

7. The processing assembly as claimed in claim 1, wherein a scraper is connected to each of two inner end faces of the tank.

8. The processing assembly as claimed in claim 1, wherein the deodorant pool has a cool water path, an inlet for introducing cool water, and an electric valve for releasing water in the deodorant pool, the inlet is connected to the vacuum pump so as to cool the vacuum pump.

9. The processing assembly as claimed in claim 8, wherein the electric valve opens when a temperature of the water in the deodorant pool is higher than 45 degrees Celsius, the water in the deodorant pool drains out from the electric valve; the inlet introduces cool water into the deodorant pool and cools down the deodorant pool.
